(19) United States
(12) Patent Application Publication
(10) Pub. No.: US 2013/0145682 A1
(43) Pub. Date: Jun. 13, 2013

(54) METHOD FOR PROCESSING A LIGNOCELLULOSIC BIOMASS MATERIAL

(71) Applicant: Shell Oil Company, Houston, TX (US)

(72) Inventors: Andrew James MURPHY, Ince (GB); David BROWN, Rijswijk (NL)

(73) Assignee: SHELL OIL COMPANY, Houston, TX (US)

(21) Appl. No.: 13/707,364

(22) Filed: Dec. 6, 2012

(30) Foreign Application Priority Data
Dec. 7, 2011 (EP) EP11192346.2

(51) Int. Cl. C12P 19/14

(52) U.S. Cl. CPC C12P 19/14
USPC 44/307; 435/99; 435/155

(57) ABSTRACT

Method for processing a lignocellulosic biomass material, comprising (a) a pretreatment process, in which the biomass is prepared for enzymatic hydrolysis, and (b) a subsequent hydrolysis process, in which the pretreated biomass is subjected to enzymatic hydrolysis of its cellulosic components to convert them into sugars, wherein the pretreatment process (a) is carried out in the presence of a tertiary polyamide additive. The additive may be used to improve the efficiency of the hydrolysis process (b). Also provided are processes for the production of a fermentation product from lignocellulosic biomass, and/or for the production of a biofuel or biofuel component.

### CROSS REFERENCE TO RELATED APPLICATIONS

This application claims the benefit of European Patent Application No. 11192346.2, filed on Dec. 7, 2011, the disclosure of which is incorporated by reference herein in its entirety.

### FIELD OF THE INVENTION

Embodiments of the present invention generally relate to a method for processing of a lignocellulosic biomass material, and more particularly to a method for generating a sugar and/or production of a fermentation product, such as a bio-alcohol, from a lignocellulosic biomass material using a tertiary polyamide additive.

### BACKGROUND TO THE INVENTION

This section is intended to introduce various aspects of the art, which may be associated with exemplary embodiments of the present invention. This discussion is believed to assist in providing a framework to facilitate a better understanding of particular aspects of the present invention. Accordingly, it should be understood that this section should be read in this light, and not necessarily as admissions of any prior art.

Alcohols such as ethanol can be used as fuels, in particular as components of gasoline fuel formulations. So-called “bioalcohols,” which have been produced from purely biological sources such as plants, can be used as biofuels. The demand for such fuels is increasing rapidly, both in the interests of the environment and to comply with increasingly stringent regulatory demands and consumer expectations.

Bioalcohols such as bioethanol can be produced by fermentation of starches and sugars, for example from food crops such as corn and sugar cane. They can also be produced from more complex plant products, by fermentation of sugars derived from them. In this case, the raw plant material needs to be treated before fermentation can begin, in order to break down more complex carbohydrates such as cellulose and hemicellulose into sugars which can undergo the fermentation reaction (also referred to as saccharification). Such treatment may involve enzymatic hydrolysis using a cellulase enzyme.

The enzymatic hydrolysis may itself also need to be preceded by a “pretreatment” step designed to break down a biomass macrostructure and disrupt the crystalline structure of the celluloses present. This pretreatment enhances enzyme accessibility to the cellulose during the subsequent hydrolysis, and can render the biomass more digestible.

Unprocessed plant material contains cellulose and lignin, and optionally hemicelluloses, and is hence also referred to as “lignocellulosic biomass.” Lignin is a complex biopolymer which forms an integral part of secondary cell walls in plants. It is hydrophobic. Possibly because of its cross-linking with other cell wall components, it can physically obstruct enzyme access to cellulose and hemicellulose. Lignin is also believed to hinder enzyme action through catalytically unproductive binding between the enzyme and the lignin, which reduces the availability of the enzyme for catalyzing cellulose conversion.

Hence, lignin is associated with reduced digestibility of plant biomass, and in turn with reduced efficiency and yield in an overall process for generating an alcohol from a biomass.

The problems associated with lignin make it necessary to use relatively high enzyme concentrations in order to achieve satisfactory yields in the biomass-to-alcohol conversion process. This may prevent the use of such processes on a commercial scale. Lignin-enzyme binding can also make it difficult to recycle the enzyme, which can again reduce the efficiency of the process and increase costs.

In the past, attempts have been made to reduce the hindering effects of lignin, either by modifying its properties or by removing it altogether. Processes for removing lignin, however, have tended to be complex and costly. Recent research has instead focused on treating the biomass in a way that modifies rather than removes the lignin which is present in it.

Prior art treatments have involved the addition of surfactants. Nonionic surfactants in particular, for example ethylene oxide polymer surfactants such as Tween™ and Triton™, have been found to increase the efficiency of enzyme hydrolysis, as have polyethylene oxides (also known as polyethylene glycols or PEGs). It is believed that these materials are able to disrupt unproductive lignin-enzyme binding, possibly by themselves adsorbing to the lignin. Other mechanisms have been suggested for the beneficial effects of surfactants, for example that they can make the substrate structure more accessible to enzymes; that they can stabilize enzymes and prevent them from being denatured during hydrolysis; that they can increase positive interactions between the cellulose substrate and the enzymes; and that they can improve lignin solubility and in that way reduce lignin-enzyme binding.

WO-A-2009/095781 describes the use of a PEG or surfactant as an additive during the hydrolysis of pretreated lignocellulosic biomass with cellulytic enzymes, wherein the hydrolysis mixture comprises >20% dry matter. WO-A-2008/134037 describes a method for digesting a lignocellulosic biomass comprising treating a lignocellulosic biomass with a surfactant; incubating the surfactant treated lignocellulosic biomass with an enzyme. It also refers to the pretreatment of lignocellulosic biomass with surfactants, in order to increase its enzymatic digestibility and the downstream recovery of glucose. Suitable surfactants are said to include both ionic and nonionic surfactants: those specifically mentioned include nonionic Tween™ surfactants; PEG; dodecyl benzene sulphonic acid (DDBSA); and glusopone surfactants.

Sewalt et al, in J Agric Food Chem 1997, 45: 1823-1828, describe the use of nitrogen-containing compounds such as polyvinyl pyrrolidone (PVP), ovalbumin and gelatin to reverse enzyme inhibition by lignin during the hydrolysis of cellulose in a filter paper substrate.

Bovine serum albumin (BSA) has also been used as an additive during the enzymatic hydrolysis of lignocellulosic biomass, and has again been found to improve the efficiency and yield of the reaction. It too is believed to adsorb to the lignin and hence to hinder lignin-enzyme binding. It is referred to as a potential further hydrolysis additive in WO-A-2008/134037.

It would be desirable to provide an improved and/or more economic method for reducing the enzyme-hindering effects of lignin, and thus for improving the efficiency of the enzymatic hydrolysis of lignocellulosic biomass. For example it would be an advancement in the art to provide a method which could overcome or at least mitigate the need for additives to be incorporated at the hydrolysis stage. In addition, it would be an advancement in the art to provide a method that allows one to use an additive that is more active. A more active additive may allow one to use a lower amount of additive and/or to decrease the time over which the additive needs to be used. This may have an advantage especially during commercial operation when the time needed for a process step and/or the amount of additive needed during a process step may have large effects on the economy of such a process step.

### SUMMARY OF THE INVENTION

According to a first aspect of the present invention there is provided a method for the generation of a sugar from lignocellulosic biomass, the method comprising:

(a) a pretreatment process, in which the biomass is prepared for enzymatic hydrolysis, and
(b) a subsequent hydrolysis process, in which the pretreated biomass is subjected to enzymatic hydrolysis of one or more of its cellulosic components to convert it into a sugar,
wherein the pretreatment process (a) is carried out in the presence of an additive which is a tertiary polyamide.

The tertiary polyamide is therefore herein also referred to as tertiary polyamide additive. In an embodiment, the hydrolysis process (b) may also be carried out in the presence of the tertiary polyamide additive.

Surprisingly, the tertiary polyamide was effective during the pretreatment.

It has surprisingly been found that the addition of a tertiary polyamide to lignocellulosic biomass, during a pretreatment step which precedes its enzymatic hydrolysis, can significantly enhance the efficiency of the hydrolysis process. This can lead to improved yields of the sugars which result from hydrolysis of cellulosic components of the biomass. In certain embodiments, the polyamide is thus effective as a pretreatment additive.

In cases, the improvement in efficiency can be sufficient to allow the hydrolysis to be carried out without the addition of for example polyethylene glycols (PEGs), surfactants, or binding proteins such as bovine serum albumin (BSA), ovalbumin or gelatin, as are required in many of the prior art biomass treatment processes. In accordance with certain aspects of the invention, a change to the biomass pretreatment process (i.e., the inclusion of a tertiary polyamide additive) can continue to exert a beneficial effect even on the downstream processes which the biomass undergoes.

Without wishing to be bound by this theory, it is believed that the tertiary polyamide additive binds to lignin present in the biomass, thus preventing the lignin from binding to the enzyme catalyst during hydrolysis and in turn leaving more of the enzyme available for productive catalysis of the cellulose substrate. The polyamide appears able to remain bound in this way downstream of the pretreatment process, for example during subsequent washing of the biomass and during its subsequent hydrolysis.

It is possible that the higher temperatures which are preferably used during biomass pretreatment can increase the possibility and/or the strength of this binding between polyamide and lignin, thus making it suitable to include the additive during the pretreatment process (a) instead of, or at least in addition to, the hydrolysis process (b).

Embodiments of the present invention may allow the use of milder conditions during the pretreatment of lignocellulosic biomass. Although the effectiveness of a pretreatment process will depend on a combination of factors such as the temperature, pressure and pH to which the biomass is exposed and the duration of that exposure, it is usually desirable for economic and other reasons to use as mild a combination of pretreatment conditions as possible, and thus to reduce the need for specialist equipment and skilled operators and also the energy input requirements. Since embodiments of the invention can improve the effectiveness of the combined pretreatment and hydrolysis processes, in terms of the efficiency of the hydrolysis, it can allow the use of milder pretreatment conditions without, or without undue, detriment to the efficiency of the overall process. In other words, the use of an appropriate additive could help to compensate for a reduction in severity of pretreatment conditions.

As illustrated in the examples, the use of a tertiary polyamide (such as polyvinyl pyrrolidone (PVP)) as an additive in embodiments of the invention may further allow one to use lower amounts of additive and/or shorter pretreatment and/or enzymatic hydrolysis periods, when compared to prior art additives such as Tween™ 20. The use of lower amounts of additive and/or shorter pretreatment and/or enzymatic hydrolysis periods may have economic advantages, especially when certain embodiments are carried out at a commercial scale.

A method according to the first aspect of the invention may be carried out in order to prepare the lignocellulosic biomass for a subsequent fermentation process. Thus, according to a second aspect, embodiments of the invention provides a process for the production of a fermentation product from lignocellulosic biomass, the process comprising subjecting the biomass to a method according to the first aspect of the invention, and subsequently inducing fermentation of a sugar from the resultant treated biomass.

In this respect, the tertiary polyamide additive may have a further advantage as it may suitably be simultaneously useful as a nitrogen-containing nutrient for any yeasts during any fermentation step. In a preferred embodiment the tertiary polyamide is therefore not removed from the product of step (a) and carried over through step (b) into any subsequent fermentation step.

A third aspect of the invention provides a process for the production of a biofuel or biofuel component, the process comprising subjecting lignocellulosic biomass to a method according to the first aspect of the invention and/or a process according to the second aspect.

Other features of embodiments of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

According to one aspect, there is provided a method for processing a lignocellulosic biomass material, the method comprising the steps of pretreating a biomass material in the presence of a tertiary polyamide additive, said biomass material comprising one or more cellulosic components; and subjecting the pretreated biomass material to enzymatic hydrolysis of the one or more cellulosic components to produce a sugar.

In one embodiment, the pretreating step is carried out at a temperature of from about 100 to 200° C. In another embodiment, the pretreating step comprises contacting the biomass material with an acid. In another embodiment, the tertiary polyamide additive is an amorphous polymer. In another embodiment, the tertiary polyamide additive exists in an amorphous form during at least a portion of at least one step of the method. In another embodiment, the tertiary polyamide additive is an amphiphilic polymer. In yet another embodiment, the tertiary polyamide additive is a polymer having one or more amphiphilic molecular regions.

In one embodiment, the tertiary polyamide additive is selected from the group consisting of polyvinyl pyrrolidones, poly(alkyl oxazolines), and any combination thereof. In another embodiment, the molecular weight of the tertiary polyamide is from about 5 to 100 kDa.

In one embodiment, the method further comprises the step of inducing fermentation of the produced sugar. In one embodiment, the fermentation produces an alcohol. In another embodiment, the method further comprises the step of incorporating a product of the fermentation into a biofuel or biofuel component. In one embodiment, the method further comprises the step of modifying the fermentation product to make it suitable for use in or as a biofuel. In one embodiment, the modifying step comprises combining the fermentation product with one or more additional fuel components to produce a fuel formulation.

In one embodiment, the method of claim 1 wherein the amount of at least one other additive is reduced as compared to a method carried out in absence of the tertiary polyamide additive. In another embodiment, the method of claim 1 wherein at least one operating condition is less severe as compared to a method carried out in absence of the tertiary polyamide additive. In another embodiment, the at least one other additive is selected from the group consisting of a surfactant, a substances having an ethylene oxide group, a protein, a nitrogen-containing compound, and any combination thereof. In another embodiment, the at least one other additive is an additive configured to be used in the enzymatic hydrolysis process.

In one embodiment, the pretreating step is carried out at a pressure of from about 0.05 to 5 MPa. In one embodiment, the tertiary polyamide additive is in a concentration of at least 0.1% w/w. In another embodiment, the hydrolysis step further comprises adding more tertiary polyamide additive to the biomass material.

### DETAILED DESCRIPTION OF PREFERRED EMBODIMENTS OF THE INVENTION

In the context of the present invention, the term “lignocellulosic biomass” refers at least to an organically derived (preferably plant-derived) material or any combination thereof, which contains both celluloses and lignin. The lignocellulosic biomass is herein also referred to as “biomass”. The biomass may for instance comprise cellulose, one or more hemicelluloses, and lignin, the cellulose and hemicelluloses representing the majority of its carbohydrate content. The biomass may for example contain about 5% w/w or more of lignin. It may contain water. It may be used, in a method according to certain aspects of the invention, in the form of a mixture of the organically derived material(s) with water and/or one or more other solvents.

The biomass may derive from any suitable source, including processed sources such as paper. It may for example comprise, or is derived from, a material selected from wheat straw, corn stover, rice straw, bagasse, corn fibre, corn cobs, wood bulk, nut shells, grasses such as switchgrass and miscanthus, paper, cotton seed hairs, plant material from sorted refuse, and any combination thereof. In one embodiment, the biomass comprises, or is derived from, a material selected from wheat straw, corn stover, bagasse, wood, grasses, and mixtures thereof. In another embodiment, it comprises wheat straw.

Prior to carrying out certain embodiments of the invention, the lignocellulosic biomass may have been treated for example to reduce the size of particles or other solid elements contained within it, and/or to increase its homogeneity. It may for example have been shredded, milled, ground and/or compacted. Instead or in addition, the pretreatment process (a) may include such a size reduction and/or homogenisation step, as described below.

The sugar which is generated from the lignocellulosic biomass using the method of the invention may be a fermentable sugar, for example selected from hexoses, pentoses, glucose and mixtures thereof. In a preferred embodiment, the sugar generated from the lignocelluosic biomass comprises glucose. Step (a) of the method according to the invention comprises a pretreatment process, in which the biomass is prepared for enzymatic hydrolysis. The biomass may suitably be a lignocellulosic biomass as described above. Step (a) suitably produces a product comprising a pretreated biomass.

“Pretreatment” or “pretreating’ of lignocellulosic biomass refers at least to a physical and/or chemical treatment which renders a cellulosic component of the biomass more accessible to an enzyme that converts carbohydrate polymers into fermentable sugars during a subsequent hydrolysis process, and/or which renders the physical structure of the biomass more susceptible to such enzymatic hydrolysis. The conditions for the pretreatment are suitably chosen in order to achieve this objective. For example, pretreatment may involve mechanical size reduction (for example by milling or grinding) and/or chemical treatment such as with an acid. Pretreatment may involve the application of elevated temperatures and/or pressures.

Apart from the presence of the tertiary polyamide additive, the pretreatment process (a) may be carried out in any desired manner. It may for instance be carried out at any suitable temperature. In a preferred embodiment, it is carried out at a temperature of equal to or more than about 90° C., more preferably equal to or more than about 100° C., even more preferably equal to or more than about 125° C. and most preferably equal to or more than about 150° C. The temperature is preferably equal to or less than about 300° C., more preferably equal to or less than about 280° C., even more preferably equal to or less than about 250° C., still more preferably equal to or less than about 200° C., even still more preferably equal to or less than about 175° C. and most preferably equal to or less than about 160° C. Hence the pretreatment may preferably be carried out at a temperature in the range from equal to or more than about 100° C. to equal to or less than about 200° C., more preferably in the range from equal to or more than about 125° C. to equal to or less than about 175° C. and most preferably in the range from equal to or more than about 140° C. to equal to or less than about 160° C.

In one embodiment, the pretreatment process comprises a mechanical size reduction and/or homogenisation process such as milling or grinding, in order to physically disrupt the macrostructure of the biomass. Where the pretreatment comprises a separate mechanical size reduction and/or homogenisation process, it need not necessarily be carried out at an elevated temperature; it could for example be carried out at ambient temperature.

The pretreatment process (a) may be carried out at any suitable pressure. It may for example be carried out at a pressure above atmospheric, for instance up to about 5 MPa (MegaPascal) or up to about 1 MPA. In a preferred embodiment the pretreatment is carried out at a pressure in the range from equal to or more than about 0.05 MPa to equal to or less than about 5 MPa, more preferably in the range from equal to or more than about 0.1 MPa to equal to or less than about 0.7 MPa, more preferably in the range from equal to or more than about 0.1 MPa to equal to or less than about 0.5 MPa.

In an embodiment, the pretreatment process (a) comprises treating the biomass with an acid, suitably at an elevated temperature as described above. This can help to solubilise hemicelluloses present in the biomass, and in cases can convert the solubilised hemicelluloses into fermentable sugars, thus removing the need for a hemicellulase enzyme during the subsequent hydrolysis process (b). Suitable acids may for example be selected from sulphuric acid, hydrochloric acid, phosphoric acid, and mixtures thereof. Sulphuric acid is most preferred. The concentration of the acid may for example be about 0.05% v/v or greater, or about 0.1% v/v or greater, or about 0.2 or 0.3 or 0.4 or 0.5% v/v or greater, in other cases about 0.75 or 1% v/v or greater. Its concentration may for example be about 5% v/v or less, or about 4 or 3 or 2% v/v or less, or about 1.5 or 1% v/v or less, or in some cases about 0.75 or 0.5% v/v or less, such as from about 0.25 to 1.5% v/v or from about 0.5 to 1.5% v/v or from about 0.25 to 0.75% v/v or from about 0.25 to 0.5% v/v.

In an embodiment, the lignocellulosic biomass is treated with an acid at a pH of from equal to or more than about 1.0 to equal to or less than about 5.0; or from equal to or more than about 0.5 to equal to or less than about 2.0; or from equal to or more than about 1.5 to equal to or less than about 2.0; such as for example from equal to or more than about 1.5 to equal to or less than about 1.8 or from equal to or more than about 1.8 to equal to or less than about 2.0. An acid treatment may be carried out before, during or after a mechanical size reduction and/or homogenisation process, in particular after.

Instead or in addition, the pretreatment process (a) may comprise a process selected from steam explosion, high pressure steam treatment, liquid hot water treatment, and any combination thereof.

The pretreatment process, or at least that part of the process which is carried out at an elevated temperature and/or in the presence of a reagent such as an acid, may have any suitable duration. It may for example be carried out for a period of about 6 seconds or more; or about 60 seconds or more; or about 5 minutes or more; or about 10 minutes or more; or about 30 minutes or more. It may be carried out for a period of up to about 3 hours, or of up to about 2 or 1 hours, for example for from about 1 to 60 minutes, or in some cases for from about 45 to 90 minutes, or for about an hour.

In one embodiment, the tertiary polyamide additive may be present during any one or more of the individual processing steps to which the biomass is subjected during the pretreatment process (a). It may for example be present during either or both of mechanical size reduction and acid treatment steps, or at least during the acid treatment step.

In another embodiment, the pretreatment process (a) may be followed by a washing step prior to the hydrolysis process (b). The lignocellulosic biomass may for example be washed with water. Instead or in addition, the pretreated biomass may be treated in order to alter its pH, in particular if all or part of the pretreatment process was carried out in an acidic environment, in preparation for the subsequent hydrolysis. For example, in another embodiment according to the invention step (a) suitably produces a product comprising a pretreated biomass and an acid such as sulphuric acid, hydrochloric acid, phosphoric acid, or a mixtures thereof; whereafter such product is treated to alter its pH and whereafter such product is used in the hydrolysis process of step (b) without washing of the product prior to such step (b). Such a method may be economically advantageous especially when applied at a commercial scale.

In certain embodiments, the tertiary polyamide additive may be any polymer which incorporates one or more tertiary polyamide groups, for example on the polymer backbone and/or on a side chain. In one embodiment, it is a copolymer of a tertiary polyamide and one or more other polymers, ie a copolymer which includes at least some tertiary polyamide groups. By a tertiary polyamide group is preferably understood a group containing two or more monomers which monomers contain one or more tertiary amides.

In a preferred embodiment, the tertiary polyamide additive is a water soluble polymer, by which is meant that it can be dissolved in water, at ambient temperature and pressure. In one embodiment, the tertiary polyamide additive can be dissolved in water to a concentration of at least about 0.1% w/w, or of at least about 0.5 or 1% w/w, such as from about 0.1 to 10% w/w. The tertiary polyamide additive is preferably capable of withstanding the conditions under which the pretreatment process is carried out, which may as described above involve an acidic pH and/or an elevated temperature.

In another embodiment, the additive is an amorphous polymer, or at least exists in amorphous form under the conditions in which it is used according to certain aspects of the invention.

In an embodiment, the additive is an amphiphilic polymer, or a polymer having one or more amphiphilic molecular regions.

The additive may be a polymer of an optionally substituted pyrrolidone: it may for example be a polyvinyl pyrrolidone (PVP) or mixture thereof. In an embodiment, the additive is a polymer of an optionally substituted oxazoline, for example a 2-alkyl oxazoline such as 2-ethyl oxazoline, or any combination of such polymers.

Thus, the additive may preferably be selected from the group consisting of polyvinyl pyrrolidones, poly(alkyl oxazolines), and any combination thereof.

The molecular weight of the tertiary polyamide may for example be about 2 kDa or greater, or about 5 kDa or greater, or in cases about 10 or 25 kDa or greater. Its molecular weight may for example be up to about 200 kDa, or up to about 150 or 100 or 75 or 50 kDa, such as from about 5 to 100 kDa or from about 5 to 75 kDa or from about 10 to 40 kDa. Preferably, the polyamide additive has a molecular weight which is similar to (for example within about 10 kDa of, or within about 8 kDa of) that of an enzyme which is used to catalyse the hydrolysis process (b).

In an embodiment of the invention, it may be preferred for the tertiary polyamide additive not to include ethylene oxide chains in its structure.

In another embodiment, it may be preferred not to include any further additives (for example surfactants, or PEGs, or proteins such as BSA) during the pretreatment process (a) and/or the hydrolysis process (b). Indeed, this may well be unnecessary due to the effects of the tertiary polyamide additive.

The tertiary polyamide additive may be present during the pretreatment process (a) at a concentration of for example about 1 mg or more per gram of the lignocellulosic biomass, or of about 5 mg or more per gram of the biomass, or of about 10 or 15 or 20 or 25 or 50 mg or more per gram of the biomass. It may for example be present at a concentration of up to about 500 mg per gram of the biomass, or of up to about 250 or 200 or 150 mg per gram of the biomass, or in cases of up to about 100 or 75 mg per gram of the biomass. A suitable concentration may for example be from about 20 to 250 mg per gram of the biomass, or from about 50 to 200 mg per gram of the biomass, such as about 50 or about 100 or about 150 mg per gram of the biomass. The optimum concentration will depend on the exact nature of the additive.

In an embodiment of the invention, because the tertiary polyamide additive is used during the pretreatment process (a), there is no need to add any or further additive during the hydrolysis process (b). It has been found, as described above, that the additive can bind to components of the biomass, in particular to lignin, and thus if added during the pretreatment it can remain with the biomass during the subsequent hydrolysis and continue to exert its beneficial effects on enzyme activity. This has been found to be the case even if the pretreated biomass is washed prior to hydrolysis.

In a specific embodiment of the invention, preferably when the polyamide additive is a polyvinyl pyrrolidone and more preferably when the additive is used at a concentration from about 40 to 120 mg per gram of biomass, the pretreatment process (a) preferably comprises treating the lignocellulosic biomass at a temperature from about 130 to 170° C. or from 140 to 160° C., and/or with a dilute acid, such as sulphuric acid, at a concentration of from about 0.1 to 1% v/v or from about 0.2 to 0.7% v/v.

Step (b) of the method according to the invention comprises a subsequent hydrolysis process, in which the pretreated biomass is subjected to enzymatic hydrolysis of one or more of its cellulosic components to convert it into a sugar.

The enzymatic hydrolysis process (b) may be carried out under any suitable conditions, which may be as known in the art. Preferably step (b) will involve contacting the pretreated biomass with a cellulytic enzyme (also referred to as cellulase enzyme) or mixture of cellulytic enzymes (i.e. a mixture of cellulases), under conditions in which the enzyme is capable of converting a cellulosic component of the biomass to one or more fermentable sugars such as hexoses, pentoses or in particular glucose. Many such enzymes and enzyme mixtures are known and commercially available, for example the Accellerase™ range of enzymes produced by Genencor and available from Sigma-Aldrich.

In particular, the hydrolysis process (b) may involve contacting the pretreated biomass with a cellulase enzyme selected from endoglucanases (which can attack regions of low crystallinity in cellulose fibres, so as to create free chain ends); exoglucanases and cellobiohydrolases (which can further degrade cellulose molecules by removing cellobiose units from the free chain ends); β-glucosidases (which can hydrolyse cellobiose to glucose); hemicellulases (which can hydrolyse hemicellulose); and any combination thereof. Accellerase™ 1000, for example, is a commercially available enzyme package which contains a mixture of such enzyme types.

The conditions under which the hydrolysis is carried out may preferably allow or promote activity of the relevant enzyme: a suitable operating temperature may for instance be from about 30 to 70° C., such as about 50° C., and a suitable operating pH may for instance be from about 4.5 to 5.5, such as about 5. The dosage at which the enzyme is used may depend on the chosen enzyme: it may for example be from about 5 to 100 mg per gram of cellulose, or from about 5 to 50 or 5 to 25 mg per gram of cellulose, or from about 10 to 20 mg per gram of cellulose, such as about 15 mg per gram of cellulose.

The hydrolysis will preferably be carried out in an aqueous environment. It may be carried out in the presence of a buffer, for example citric acid or a salt thereof. The biomass will preferably be present in water at a cellulose concentration of about 1% w/v or greater, or of about 2.5 or 5% w/v or greater. This cellulose concentration may for example be up to about 10% w/v, or up to about 7.5% w/v, such as about 5% w/v.

The hydrolysis process may have any suitable duration. It may for example be carried out for a period of about 30 minutes or more, or of about 1 or 2 or 5 or 10 hours or more, or in cases of about 25 or 50 or 70 hours or more. It may be carried out for a period of up to about 200 hours, or of up to about 170 or 150 or 100 hours, or of up to about 90 or 80 or 75 hours.

As indicated above, in step (b) preferably one or more sugars may be produced. Preferably such sugars include glucose. The one or more sugars produced in step (b) can advantageously be used in a fermentation process to produce one or more alcohols. Hence in a preferred embodiment the invention provides a method for the conversion of a lignocellulosic biomass, the method comprising:

(a) a pretreatment process, in which the biomass is prepared for enzymatic hydrolysis; and
(b) a subsequent hydrolysis process, in which the pretreated biomass is subjected to enzymatic hydrolysis of one or more of its cellulosic components to convert it into a sugar; and
(c) a subsequent fermentation process, in which the sugar is converted into an alcohol;
wherein the pretreatment process (a) is carried out in the presence of an additive which is a tertiary polyamide.

An embodiment according to the first aspect of the invention may be carried out in order to prepare the lignocellulosic biomass for a subsequent fermentation process. Thus, according to a second aspect, the invention provides a process for the production of a fermentation product from lignocellulosic biomass, the process comprising subjecting the biomass to a method according to the first aspect of the invention, and subsequently inducing fermentation of a sugar from the resultant treated biomass.

The fermentation product is preferably an alcohol, more preferably ethanol. The second aspect of the invention can therefore be used to generate a bioalcohol—which may be of use in or as a biofuel such as biogasoline—from biomass. Accordingly, a third aspect of the invention provides a process for the production of a biofuel or biofuel component, the process comprising subjecting lignocellulosic biomass to a method according to the first aspect of the invention and/or a process according to the second aspect.

Fermentation of the pretreated and hydrolysed biomass, or of a sugar component thereof, may be carried out in any conventional manner. It may in particular be catalyzed by a microorganism, more particularly a yeast such as of the Saccharomyces species.

An embodiment according to the second or the third aspect of the invention may comprise one or more additional processing steps downstream of the fermentation step. For example, the process may comprise separating out the biomass (and in particular lignin) residue from the fermentation broth, for instance by centrifugation, and/or recovering the desired fermentation product, for instance by distillation, and/or purifying the recovered fermentation product. Without wishing to be bound by any kind of theory, it is believed that since the tertiary polyamide additive appears to bind with the lignin components of biomass, it can be readily removed with the biomass residue.

An embodiment of the third aspect of the invention may comprise a processing step in which the properties of the relevant fermentation product are modified in order to make it suitable for use in or as a biofuel. In an embodiment, the process comprises the step of combining the fermentation product with one or more additional fuel components, for example gasoline fuel components and/or fuel additives, in order to produce a fuel formulation.

According to a fourth aspect, certain embodiments of the invention provide the use of a tertiary polyamide as a pretreatment additive in a method for the generation of a fermentable sugar from lignocellulosic biomass, for one or more of the following purposes:

i. improving the efficiency of the method, or of a processing step forming part of the method, in particular the hydrolysis of a cellulosic component of the biomass;

ii. improving the yield of the fermentable sugar;

iii. allowing one or more processing steps during the method (in particular a pretreatment process) to be carried out under less severe conditions, for example at a lower temperature, at a pH closer to neutral, for a shorter period of time and/or with a lower amount or concentration of a component such as an acid or an enzyme catalyst; and

iv. reducing the amount of another additive which is used in the method for one or more of the purposes (i) to (iii).

As described above in connection with the first aspect of the invention, embodiments that “use” a tertiary polyamide additive may involve pretreating the biomass with, or in the presence of, the polyamide. Pretreating the biomass with the tertiary polyamide will suitably mean contacting the biomass with the polyamide either before or during a pretreatment process, for instance by suspending the biomass in a fluid system (in particular an aqueous system) which contains the polyamide. The polyamide may be dissolved in the fluid system. It may be added to such a system before and/or during a pretreatment process to which the biomass is subjected, in particular before and/or during a chemical treatment which forms part of a pretreatment process, for example an acid treatment.

Embodiments that “use” a tertiary polyamide in the ways described above may also embrace supplying the polyamide together with instructions for its use, as a pretreatment additive in a method for generating a sugar from lignocellulosic biomass, for one or more of the purposes (i) to (iv) above. The polyamide may be supplied as part of a composition which is suitable and/or adapted and/or intended for inclusion in a biomass pretreatment process which forms part of a sugar generating method, in which case the tertiary polyamide may be included in such a composition for the purpose of influencing its effects on a method for generating a sugar from lignocellulosic biomass.

In connection with purposes (i) and (ii), certain embodiments may use the tertiary polyamide additive to achieve any degree of improvement in the efficiency or yield, as the case may be, and/or for the purpose of achieving a desired target efficiency or yield. “Achieving” a desired target property also embraces—and in an embodiment involves—improving on the relevant target. Thus, for example, the tertiary polyamide may be used to cause the yield of the fermentable sugar to exceed a desired target value.

Similarly, in connection with purpose (iii), certain embodiments may use the tertiary polyamide to achieve any degree of change in the severity of the conditions under which the relevant processing step(s) is or are carried out, and/or for the purpose of achieving a desired target change. Suitably, it is used to achieve the relevant change whilst at the same time maintaining the efficiency and/or the yield of the relevant processing step, and/or of the overall method, at or above a desired target level.

An improvement in the efficiency of the method or of a processing step may be manifested by an increase in the rate at which the method or step proceeds, at a specific point in time and/or over a specific period of time, and/or before reaching a desired target yield. Instead or in addition, it may be manifested by a reduction in the amount of energy and/or other resources (for example expense, space, manpower or equipment) needed in order to carry out the method or processing step for a desired time period or in order to generate a desired target yield. It may be manifested by a reduction in the severity of the conditions needed in order to carry out the method or processing step at a desired rate and/or to a desired outcome—for example, it may mean that a processing step can be carried out at a lower temperature.

An improvement in the yield of the fermentable sugar may be manifested by a higher yield in any one or more fermentable sugars, at a specific point in time and/or over a specific period of time and/or on completion of the method or of a hydrolysis step which forms part of the method.

In connection with purpose (iv), certain embodiments may use the tertiary polyamide to achieve any degree of reduction in the amount of the other additive, and/or for the purpose of achieving a desired target reduction in that amount. The term “reducing” also embraces reduction to zero. The reduction may for instance be about 10% or more of the original additive amount, or about 25 or 50 or 75 or 90% or more. The reduction may be as compared to the amount of the other additive which would otherwise have been used in the method in order to achieve a desired efficiency and/or yield. This may for instance be the amount of the other additive which was used in one embodiment, and/or which was used in an otherwise analogous method, prior to the realisation that a tertiary polyamide could be used in the way provided by certain aspects of the present invention, or prior to using a tertiary polyamide in the method in accordance with certain aspects of the invention.

The reduction in the amount of the other additive may be as compared to the amount of the other additive which would be predicted to be necessary in order to achieve a desired target efficiency and/or yield for the method in the absence of the tertiary polyamide.

In connection with purpose (iv), the other additive may for example be selected from surfactants (in particular nonionic surfactants); substances having ethylene oxide groups (for example PEGs); proteins such as BSA, ovalbumin or gelatin; nitrogen-containing compounds; and mixtures thereof. The other additive may be an additive which is used or intended to be used in a pretreatment and/or a subsequent enzymatic hydrolysis process to which the biomass is subjected, in particular in an enzymatic hydrolysis.

In accordance with the fourth aspect of the invention, an embodiment in which the tertiary polyamide is used may itself form part of an overall process for the production of a fermentation product (in particular an alcohol) from lignocellulosic biomass. It may form part of an overall process for the production of a biofuel or biofuel component, in particular a bioalcohol. In these contexts, the tertiary polyamide may be used for the purpose of improving the efficiency and/or the yield of the overall process, and/or for allowing one or more of the process steps to be carried out under less severe conditions, and/or for reducing the amount of another additive which is present during the process.

In accordance with the invention, a tertiary polyamide may be marketed with an indication that it can be used, as a pretreatment additive in a method for generating a sugar from lignocellulosic biomass, for one or more of the purposes listed above in connection with the fourth aspect of the invention. The marketing of a tertiary polyamide may include an activity selected from (a) providing the polyamide in a container that comprises the relevant indication; (b) supplying the polyamide with product literature that comprises the indication; (c) providing the indication in a publication or sign (for example at the point of sale, including on the internet) that describes the polyamide; and (d) providing the indication in a commercial which is aired for instance on the radio, television or internet.

Furthermore, a method for generating a sugar from lignocellulosic biomass, or any other method for the treatment of lignocellulosic biomass which involves subjecting the biomass to an enzymatic hydrolysis, may be marketed with an indication that it benefits from an improvement due to the use of a tertiary polyamide as a pretreatment additive in the method, in particular a greater efficiency. The marketing of such a method may include an activity selected from (a) carrying out the method, or offering the method for use by another party, together with product literature that comprises the indication; (b) providing the indication in a publication or sign (for example at the point of sale, including on the internet) that describes the method; and (c) providing the indication in a commercial which is aired for instance on the radio, television or internet. The improvement may be attributed, in such an indication, at least partly to the presence of the tertiary polyamide as a pretreatment additive. The invention may involve assessing the relevant property (in particular the efficiency and/or yield) while or after the method is carried out. It may involve assessing the relevant property both with and without the tertiary polyamide, for example so as to confirm that the polyamide contributes to the relevant improvement in the method.

Throughout the description and claims of this specification, the words “comprise” and “contain” and variations of the words, for example “comprising” and “comprises”, mean “including but not limited to”, and do not exclude other moieties, additives, components, integers or steps. Moreover the singular encompasses the plural unless the context otherwise requires: in particular, where the indefinite article is used, the specification is to be understood as contemplating plurality as well as singularity, unless the context requires otherwise.

Preferred features of each aspect of the invention may be as described in connection with any of the other aspects. Other features of the invention will become apparent from the following examples. Generally speaking the invention extends to any novel one, or any novel combination, of the features disclosed in this specification (including any accompanying claims and drawings). Thus features, integers, characteristics, compounds, chemical moieties or groups described in conjunction with a particular aspect, embodiment or example of the invention are to be understood to be applicable to any other aspect, embodiment or example described herein unless incompatible therewith. Moreover unless stated otherwise, any feature disclosed herein may be replaced by an alternative feature serving the same or a similar purpose.

Where upper and lower limits are quoted for a property, for example for the concentration of a fuel component, then a range of values defined by a combination of any of the upper limits with any of the lower limits may also be implied.

In this specification, references to properties such as solubilities and liquid phases are—unless stated otherwise—to properties measured under ambient conditions, ie at atmospheric pressure and at a temperature of from 16 to 22 or 25° C., or from 18 to 22 or 25° C., for example about 20° C.

The present invention will now be further described with reference to the following non-limiting examples.

### Example 1

This example demonstrates the ability of a tertiary polyamide pretreatment additive to enhance the saccharification of lignocellulosic plant biomass.

Wheat straw was pretreated both in the presence and the absence of a tertiary polyamide additive. An enzymatic hydrolysis was then performed on both a washed and an unwashed portion of each of the pretreated samples. The extent of hydrolysis was determined by the amount of glucose liberated. All experiments were performed in triplicate.

The wheat straw was from a source harvested in autumn 2009 in Texas, USA. The additive was polyvinyl pyrrolidone (PVP) of molecular weight 40 kDa, ex Sigma-Aldrich, and was used in the pretreatment process at a concentration of 50 mg per gram of biomass.

Pretreatment involved two steps: size reduction and homogenisation of the biomass, followed by treatment with acid at an elevated temperature. The first step was performed using a bench scale ball mill (Retsch™ MM 400) to reduce the wheat straw to a powder (grinding time 1 minute; vibrational frequency 30 Hz). The resultant particles had a size of approximately 5 μm. The milled wheat straw was stored at room temperature until the second, acid treatment, step.

The milled straw was then slurried in dilute sulphuric acid (1% v/v) with a ratio of 1 g of biomass to 9 mL of the aqueous acid solution. Where appropriate, the PVP was added to this slurry. The pretreatment was performed at 125° C. for 1 hour, in an autoclave. After pretreatment, the substrate was filtered, and where appropriate washed in water to remove soluble material.

The subsequent enzymatic hydrolysis was performed in 250 mL sealed flasks with 5% cellulose (grams of cellulose per 100 mL of slurry) in 50 mM citric acid buffer at pH 5. The enzyme used was Accellerase™ 1000 (ex Sigma-Aldrich), at a concentration of 15 mg of enzyme per gram of cellulose. The reaction was initiated by mixing the buffer containing the enzyme, preheated to 50° C., with the solid pretreated biomass substrate which had also been preheated to 50° C. The reaction mixture was incubated at 50° C. for 72 hours in a shaker incubator (Infors HT Multitron™) at 250 rpm.

In the case of unwashed pretreated samples, the hydrolysis was preceded by neutralisation to pH 5 using a solution of 4N sodium hydroxide (NaOH).

Glucose concentrations were determined by high performance liquid chromatography (HPLC) from 1 mL aliquots taken from the reaction mixture at appropriate time points during or after the hydrolysis. The aliquots were centrifuged at 13000 g for 1 minute, immediately after removal from the reaction mixture. 100 μL of the resultant supernatant was then diluted in 900 μL of 10 mM sulphuric acid to stop the hydrolysis, followed by HPLC analysis using a Bio-Rad Aminex™ HPX-87P column. Percentages of cellulose conversion were calculated from the measured glucose levels and the cellulose content of the original substrate, the latter being determined from the maximum amount of glucose that could be liberated by completely hydrolysing the cellulose.

The results are shown in Table 1 below.

TABLE 1

| | % cellulose |
|---|---|
| Sample | conversion |
| 1A - pretreated without PVP; washed | 40.4 |
| 1B - pretreated with PVP; washed | 60.9 |
| 1C - pretreated without PVP; unwashed | 32.7 |
| 1D - pretreated with PVP; unwashed | 51.0 |

These results show that the PVP pretreatment additive improved the saccharification of the washed and pretreated wheat straw by more than 50%. For the unwashed samples (more reflective of an industrial process), the addition of PVP during pretreatment increased cellulose conversion by almost 56%.

Of note is that the improvement persists even for the washed samples. Thus, it appears to be sufficient to use the additive during the pretreatment step alone. Since washing might be expected to remove the water-soluble PVP, this suggests that the polymer may be interacting in some way with the biomass during the pretreatment, remaining bound to the biomass through the subsequent washing step and going on to affect the downstream hydrolysis.

### Example 2

This example provides more information about the effects of a tertiary polyamide additive during both pretreatment and hydrolysis of lignocellulosic biomass.

Experiments were performed using the same basic methodology as in Example 1, as follows;

Sample 2A: pretreatment without PVP+hydrolysis without PVP

Sample 2B: pretreatment with PVP+hydrolysis without PVP

Sample 2C: pretreatment without PVP+hydrolysis with PVP

Sample 2D: pretreatment with PVP+hydrolysis with PVP

The enzymatic hydrolysis was carried out for 73 hours. The PVP additive was dosed at 50 mg per gram of biomass, whether at the pretreatment and/or the hydrolysis stage. The pretreated biomass was washed prior to the hydrolysis step.

Samples were removed at intervals during the enzymatic hydrolysis (T=23, 46 and 73 hours from the start), and assayed for glucose levels. The results are shown in Table 2 below, again as percentages of cellulose conversion during the hydrolysis step. All experiments were performed in triplicate; SD stands for standard deviation.

TABLE 2

| | % cellulose conversion | | | | | |
|---|---|---|---|---|---|---|
| | T = 23 | SD | T = 46 | SD | T = 73 | SD |
| Sample | hours | (n = 3) | hours | (n = 3) | hours | (n = 3) |
| 2A | 33.3 | 0.42 | 38.3 | 0.52 | 41.6 | 0.46 |
| 2B | 44.7 | 0.27 | 54.6 | 0.32 | 60.9 | 0.22 |
| 2C | 40.2 | 0.40 | 49.4 | 0.59 | 54.5 | 0.55 |
| 2D | 45.9 | 0.77 | 55.9 | 0.19 | 62.4 | 0.47 |

Table 2 shows that the hydrolysis proceeded at a significantly faster rate when the biomass had been pretreated with the PVP than when it had received no PVP treatment (compare samples 2A and 2B). The addition of PVP only during the hydrolysis also increased the rate of conversion to glucose, but less so than when PVP was present during pretreatment (compare samples 2A, 2B and 2C). The use of PVP during both pretreatment and hydrolysis had a relatively small effect on conversion rate compared to the use of PVP during pretreatment only (samples 2B and 2D). Thus, in accordance with the invention the tertiary polyamide additive can provide particular benefits when included during pretreatment.

The relatively small difference between the results for samples 2B and 2D suggests that there is no difference in the mechanism of action of the additive when it is incorporated during the hydrolysis as opposed to during pretreatment, but it does appear that the conditions to which the additive is exposed during pretreatment may contribute to its effectiveness downstream. It is possible that the elevated temperatures used during pretreatment may influence the ability of the polyamide to bind with lignin in the biomass.

### Example 3

This example shows the effect of additive concentration in one embodiment of the invention. Experiments were performed using the same basic methodology as in Example 1, using three different concentrations of the PVP additive during the pretreatment process: 10, 50 and 100 mg per gram of biomass. A control sample had no PVP added. The enzymatic hydrolysis was carried out for 73 hours, on washed biomass. All experiments were performed in triplicate.

The results are shown in Table 3 below. The figures quoted are for the measured improvement in percent cellulose conversion, relative to that observed for the control sample with no PVP.

TABLE 3

| | % improvement in | SD |
|---|---|---|
| Sample | cellulose conversion | (n = 3) |
| 3A - no PVP (control) | 0 | 0.32 |
| 3B - 10 mg/g PVP | 9.9 | 0.65 |
| 3C - 50 mg/g PVP | 50.9 | 0.22 |
| 3D - 100 mg/g PVP | 71.3 | 0.62 |

The improvement in cellulose conversion efficiency initially increased with increasing polyamide concentration, in an approximately linear fashion. However, above about 50 mg/g PVP, the improvement appeared to approach a plateau. Similar trends have been observed for other additives such as PEG and the nonionic surfactant Tween™. The effect may suggest that the PVP works by binding to lignin present in the biomass, the plateau potentially reflecting saturation of lignin binding sites. If that is the case, then when carrying out the invented method, it may be possible to limit additive concentrations to those which represent saturation of lignin binding sites.

### Example 4

This example illustrates the effects of pretreatment acidity and pretreatment temperature on the effectiveness of a tertiary polyamide additive.

Experiments were performed using the same basic methodology as in Example 1, but using a range of pretreatment temperatures (125, 150 and 175° C.) and a range of sulphuric acid concentrations (0, 0.125, 0.25, 0.375 and 0.5% v/v). For each pair of treatment conditions, samples were pretreated both with and without PVP.

The pretreatments were carried out for 1 hour in a 5000 Series Multiple Reactor System (Parr Instrument Company). The enzymatic hydrolysis was carried out for 72 hours, after washing the pretreated samples. The results are shown in Table 4 below.

TABLE 4

| | Pretreatment | Sulphuric acid | | |
|---|---|---|---|---|
| | temperature | concentration | PVP | % cellulose |
| Sample | (° C.) | (% v/v) | present? | conversion |
| 4A | 125 | 0 | No | 14.3 |
| 4B | 125 | 0 | Yes | 15.9 |
| 4C | 125 | 0.125 | No | 24.3 |
| 4D | 125 | 0.125 | Yes | 28.5 |
| 4E | 125 | 0.25 | No | 36.3 |
| 4F | 125 | 0.25 | Yes | 44.7 |
| 4G | 125 | 0.375 | No | 40.4 |
| 4H | 125 | 0.375 | Yes | 51.4 |
| 4J | 125 | 0.5 | No | 48.7 |
| 4K | 125 | 0.5 | Yes | 54.9 |
| 4L | 150 | 0 | No | 17.4 |
| 4M | 150 | 0 | Yes | 22.6 |
| 4N | 150 | 0.125 | No | 36.3 |
| 4P | 150 | 0.125 | Yes | 50.1 |
| 4Q | 150 | 0.25 | No | 45.6 |
| 4R | 150 | 0.25 | Yes | 68.9 |
| 4S | 150 | 0.375 | No | 45.8 |
| 4T | 150 | 0.375 | Yes | 71.6 |
| 4U | 150 | 0.5 | No | 46.9 |
| 4V | 150 | 0.5 | Yes | 67.4 |
| 4W | 175 | 0 | No | 39.3 |
| 4X | 175 | 0 | Yes | 46.1 |
| 4Y | 175 | 0.125 | No | 45.3 |
| 4Z | 175 | 0.125 | Yes | 70.4 |
| 4AA | 175 | 0.25 | No | 43.4 |
| 4AB | 175 | 0.25 | Yes | 51.5 |
| 4AC | 175 | 0.375 | No | 37.7 |
| 4AD | 175 | 0.375 | Yes | 36.6 |
| 4AE | 175 | 0.5 | No | 33.9 |
| 4AF | 175 | 0.5 | Yes | 43.4 |

For virtually all of the samples, the presence of PVP during pretreatment improved the efficiency of the subsequent hydrolysis. This confirms that PVP is effective as a pretreatment additive over a range of operating temperatures and pHs.

The improvement was generally more marked when the pretreatment was carried out at 150° C. than at the other two temperatures: under these conditions, improvements of up to 56% were observed in the cellulose conversion. That said, a good improvement was also seen at 175° C. and 0.125% v/v sulphuric acid, suggesting perhaps that a higher temperature (for example higher than 150° C., or than 160 or 170° C.) might be best combined with a lower acid concentration (perhaps 0.3% v/v or lower, or 0.25 or 0.2 or 0.15% v/v or lower).

The addition of acid on the whole appeared to have a beneficial effect on the process, with increasing acid concentration leading in general to increases in cellulose conversion. This may be explained by the fact that the acid can increase hemicellulose removal and therefore facilitate the conversion of cellulose to glucose.

The data obtained at 175° C. without PVP indicated a slight improvement in cellulose conversion with 0.125% v/v sulphuric acid. Higher acid concentrations led to decreases in cellulose conversion. This may indicate that at higher temperatures the biomass may be “overcooked”, and that a shorter pretreatment residence time (for example an hour or less, or 30 minutes or less) may be more appropriate at such temperatures and pHs. Indeed, high temperature and acid concentration are known to lead to changes in the structure of cellulose fibres, which can reduce their accessibility to cellulytic enzymes.

The results obtained at 175° C. with PVP were less consistent. The maximum cellulose conversion was 70.4% with 0.125% v/v sulphuric acid. At higher acid concentrations, cellulose conversion reduced, again most likely due to overcooking.

### Example 5

This example demonstrates the effect of a tertiary polyamide additive on a fermentation process downstream of the pretreatment and enzymatic hydrolysis steps.

Wheat straw was pretreated, and subsequently subjected to enzymatic hydrolysis, using the same basic methodology as in Example 1. Sample 5A was pretreated in the presence, and sample 5B in the absence, of the PVP additive. Both were neutralised at pH 5 using 4N aqueous sodium hydroxide, but not washed, between the pretreatment and hydrolysis steps. The hydrolysis was carried out for 72 hours.

The wheat straw hydrolysates were filtered, and then fermented using the yeast Thermosacc Dry™ (Lallemand Ethanol Technology), which is an active dry strain of Saccharomyces cerevisiae.

To prepare the yeast inoculums, solutions containing 10 g/L of the yeast were prepared by solubilising 1 g of Thermosacc Dry™ in 100 mL YPD media (YPD contains 20 g/L peptone, 20 g/L glucose and 10 g/L yeast extract). Yeast propagation was initiated by inoculating two baffled 1 litre shake flasks, each containing 500 mL of fresh yeast medium, with 2.5 mL of the previous culture in YPD. The two flasks were sealed with sterile cotton wool to allow aeration of the samples, and placed overnight (18 hours) inside an orbital shaking incubator (Stuart™ SI500) set at 30° C. and 160 rpm, in order to reach a final concentration of 7-10 g/L yeast in each flask. Next, two 500 mL centrifuge tubes were filled from the two baffled flasks, and centrifuged at 4000 g for 5 minutes. The resultant supernatants were then discarded, and the pellets thoroughly re-suspended in fresh yeast medium to a concentration of 20 g/L.

The yeast medium was a standard medium including glucose, salts (potassium dihydrogen phosphate, ammonium sulphate and magnesium sulphate), sodium acetate, metal trace elements (in the form of zinc sulphate, cobalt chloride, copper (II) sulphate, calcium chloride, iron (II) sulphate, boric acid and potassium iodide, with EDTA to maintain salt solubility) and vitamin trace elements (biotin, calcium panthothenate and cobalt chloride, in an ethanol/water mix) in deionised water. All stock solutions used in the preparation of the medium were sterilised in an autoclave prior to use. The pH of the medium was 6.0.

The fermentation step was performed batch-wise as follows. The experiments were carried out in 500 mL Duran bottles with a working volume of 400 mL using an alcohol fermentation monitor (AFM) to control the temperature, with stirring. 360 mL samples of the hydrolysed wheat straw were preheated to 37° C. in 500 mL Duran bottles after being installed on the AFM. The fermentations were initiated by injecting 20 mL of the 20 g/L yeast solution through a sample port of the AFM. Sample ports were rinsed by injecting 20 mL of fresh yeast medium into the fermentation assays, bringing the working volume to 400 mL and the yeast concentration to 1 g/L. The fermentation was carried out overnight under anaerobic conditions at 37° C. and 120 rpm.

Glucose concentrations in each sample were measured before fermentation (in both the hydrolysate and the fermentation assay) and after fermentation. Ethanol concentrations were measured before and after fermentation. Glucose and ethanol concentrations were determined by HPLC, in the manner described in Example 1.

The results are shown in Table 5 below. The figures quoted are concentrations in g/L.

TABLE 5

| | Before fermentation | | | | | | | |
|---|---|---|---|---|---|---|---|---|
| | [Glucose] | | [Glucose] in | | | After fermentation | | |
| | in | SD | fermentation | SD | | | SD | |
| Sample | hydrolysate | (n = 2) | assay | (n = 2) | [Ethanol] | [Ethanol] | (n = 2) | [Glucose] |
| 5A | 29.3 | 0.41 | 38.4 | 0.37 | 0 | 19.3 | 0.07 | 0 |
| 5B | 19.6 | 0.41 | 29.6 | 0.36 | 0 | 14.9 | 0.32 | 0 |

It can be seen that in both samples, all of the glucose was consumed and converted to ethanol during the fermentation step. The higher glucose content in sample 5A, due to its pretreatment with the tertiary polyamide, translated to a higher ethanol content following fermentation. Thus, the PVP additive appeared not to inhibit the fermentation process or the action of the yeast.

### Example 6

This example confirms the observations in Example 5, that the tertiary polyamide pretreatment additive is not detrimental to the subsequent fermentation of a pretreated biomass.

The general method of Example 5 was repeated, and carbon dioxide production was monitored during the fermentation process, using an alcohol fermentation monitor. The results are shown in Table 6 below. Sample 6A had been pretreated in the presence of PVP, sample 6B without PVP.

TABLE 6

CO₂ production (mmol/h)

| Time (hours) | Sample 6A | Sample 6B |
|---|---|---|
| 0 | 0 | 0 |
| 2 | 9.1 | 9.3 |
| 4 | 17.1 | 17.1 |
| 6 | 21.7 | 20.7 |
| 8 | 21.5 | 19.4 |
| 10 | 3.4 | 1.3 |
| 12 | 0.3 | 0.03 |
| 14 | 0.01 | 0.001 |

The rate of carbon dioxide production is directly related to the rate of ethanol production during the fermentation process. The Table 6 data therefore show that ethanol production rates were similar whether or not the biomass had been pretreated with the tertiary polyamide additive. This again confirms that the additive had no detrimental effect on the downstream conversion of the biomass hydrolysate to the end product ethanol.

### Example 7

This example shows the effect of additive molecular weight on one embodiment of the invention.

Experiments were performed using the same basic methodology as in Example 1, but using as the pretreatment additives a number of PVP polymers of differing molecular weights: 10, 40, 360 and 1300 kDa. A further experiment used the vinyl pyrrolidone monomer as a pretreatment additive, whilst a control used no pretreatment additive at all. The additives were all used at a concentration of 50 mg per gram of biomass substrate. The enzymatic hydrolysis was carried out for 73 hours.

The results are shown in Table 7 below.

TABLE 7

| | | % cellulose | SD |
|---|---|---|---|
| Sample | Pretreatment additive | conversion | (n = 3) |
| 7A | None | 41.6 | 0.46 |
| 7B | Vinyl pyrrolidone monomer | 41.0 | 0.76 |
| 7C | PVP - 10 kDa | 59.6 | 0.81 |
| 7D | PVP - 40 kDa | 60.2 | 1.00 |
| 7E | PVP - 360 kDa | 49.9 | 0.45 |
| 7F | PVP - 1300 kDa | 50.9 | 1.84 |

All of the polymeric additives caused an improvement in cellulose conversion as compared to the control sample 7A. The shorter chain polymers, of molecular weight 10 and 40 kDa, caused a greater improvement than the higher molecular weight polymers (compare samples 7C and 7D with 7E and 7F). The monomer used for sample 7B gave no improvement over the control.

Thus, polyamide chain length does appear to be capable of influencing the effectiveness of the invented method. This may support the theory that the polymer is interacting with the biomass and thereby preventing unproductive binding of enzymes to lignin. Eriksson et al (Enzyme and Microbial Technology 31: 353-364) have shown that the adsorption of Ce17A, an exoglucanase of Trichoderma reesei, was reduced during enzymatic hydrolysis by the addition of nonionic surfactants. This mechanism was explained by hydrophobic sites on lignin being occupied by the surfactant. The hydrophilic portions of the surfactant will in turn protrude into the aqueous solution and cause steric repulsion of the enzyme from the lignin surface. Like surfactants, PVP has been described as an amphiphilic molecule, and might therefore be able to bind to hydrophobic sites on lignin.

Interestingly, the molecular weight of the exoglucanase from Trichoderma reesei which is present in the Accellerase™ 1000 package is believed to be 46 kDa, similar to the molecular weight of the 40 kDa PVP used in the present experiments. It is possible that the size of the additive molecule plays a role in stabilising the binding between hydrophobic sites on lignin and hydrophobic parts of the additive molecule. Additive molecules with a much higher molecular weight, for example the 360 and 1300 kDa PVP, might form less stable interactions with lignin and thus be less effective in maintaining levels of unbound, and hence catalytically active, enzyme.

### Example 8

This example investigated potential interactions between a tertiary polyamide pretreatment additive and cellulosic components of biomass.

Three cellulose-containing samples were pretreated using the same basic methodology as in Example 1, both with and without the PVP additive. The three sources were α-cellulose, Avicel™ and filter paper. Avicel™ is a microcrystalline cellulose ex Sigma Aldrich. The filter paper (3MM Whatman) was sourced from GE Healthcare. Following pretreatment, the samples were washed and then subjected to enzymatic hydrolysis for 72 hours. The percentages of cellulose conversion are shown in Table 8 below.

TABLE 8

| | | |
|---|---|---|
| | SD | % cellulose |
| Sample | (n = 3) | conversion |

| | | | |
|---|---|---|---|
| 8A | α-cellulose - no PVP in | 1.86 | 49.0 |
| | pretreatment | | |
| 8B | α-cellulose - PVP in | 1.32 | 48.0 |
| | pretreatment | | |
| 8C | Avicel ™ - no PVP in | 1.43 | 57.2 |
| | pretreatment | | |
| 8D | Avicel ™ - PVP in | 1.45 | 57.2 |
| | pretreatment | | |
| 8E | Filter paper - no PVP in | 0.56 | 30.8 |
| | pretreatment | | |
| 8F | Filter paper - PVP in | 1.01 | 46.0 |
| | pretreatment | | |

For samples 8A to 8D, the presence or absence of PVP during the pretreatment process appeared to have little or no effect on the efficiency of the subsequent hydrolysis. This implies that the effect of PVP on the pretreatment of lignocellulosic biomass is due to some interaction with the lignin component rather than the celluloses present. Either the PVP was removed during the washing step (which would indicate that it did not bind to the cellulose), or if it survived the washing it was still not influencing enzyme access to the cellulose.

The results obtained with the filter paper sample indicated a positive effect for the PVP on cellulose conversion. However, it is believed that the low cellulose conversion observed in sample 8E (filter paper without PVP pretreatment; cellulose conversion 30.8%) can be explained by the surface properties of filter paper, which have been shown to promote protein adsorption. The high adsorption of β-glucosidase by filter paper is believed to reduce the enzyme's availability in liquid, which can cause accumulation of cellobiose. Therefore, the improvement in cellulose conversion in the presence of PVP might indicate that the PVP has a role in preventing the adsorption of enzymes to the filter paper substrate.

### Example 9

This example investigated whether a tertiary polyamide pretreatment additive serves to remove lignin from the pretreated biomass.

The method used to measure lignin content was based on the liquefaction of biomass. Experiments were carried out using the same basic methodology as in Example 1, using either PVP or Triton™ X-100 as the pretreatment additive (both at a concentration of 50 mg per gram of substrate). A control was pretreated without any additive. Triton™ X-100 is a commercially available nonionic surfactant (octyl-phenol(ethyleneglycol)-9,6-ether), which has been used in the past as a pretreatment additive to enhance enzymatic hydrolysis; it was sourced from Sigma-Aldrich.

The pretreated samples were washed and dried. The weight of the solid biomass was measured both before and after pretreatment in order to determine the percentage of liquefaction during the pretreatment. The results are shown in Table 9 below.

TABLE 9

| | | % | SD |
|---|---|---|---|
| Sample | | lique-faction | (n = 3) |
| 9A | Pretreated without additive | 43.7 | 0.20 |
| 9B | Pretreated with PVP | 39.3 | 0.18 |
| 9C | Pretreated with Triton ™ X-100 | 40.1 | 0.65 |

Of note is that the percentage of liquefaction was slightly higher for the unadditivated pretreatment. This indicates that neither the surfactant nor the polyamide additive acts by removing biomass, although they may be interacting with the biomass in some way which may explain the approximately 5% difference in the amount of liquefaction between the additivated and unadditivated samples. Of particular note is that the present invention can result in improvements in enzymatic hydrolysis, without actually removing lignin. This provides further support for the theory that the tertiary polyamide additive is instead modifying the properties and/or behaviour of the lignin, most probably by binding to it.

### Example 10

This example investigated further the interaction between a tertiary polyamide pretreatment additive and the components of lignocellulosic biomass.

Samples of wheat straw, Avicel™ and filter paper were pretreated using the same basic methodology as in Examples 1 and 8, using various PVP concentrations. A control sample was pretreated without any additive. The pretreated samples were then washed, filtered, frozen at −80° C. and dried overnight using a freeze-dryer. They were exposed to ambient air for approximately half a day to allow them to stabilise, before undergoing a CHN analysis using an Organic Elemental Analyzer (Flash 2000, Brechbühler).

CHN analysis (Carbon, Hydrogen and Nitrogen analysis) is a form of elemental analysis that allows simultaneous determination of the carbon, hydrogen and nitrogen contents of a sample by means of high temperature combustion of a microgram-sized sample in an oxygen-enriched helium atmosphere. The combustion products, which are elemental gases (CO₂, NOₓ (reduced to N₂) and H₂O), are separated in a chromatographic column and finally detected by a highly sensitive thermal conductivity detector. The results are reported as weight percentages for each element.

The nitrogen contents of the samples are shown in Table 10 below. The PVP concentrations are expressed in mg per gram of cellulose. The CHN analyses were performed in triplicate.

TABLE 10

| | | Nitrogen | SD |
|---|---|---|---|
| Material | Pretreatment | content (%) | (n = 3) |
| Wheat straw | No additive | 0.13 | 0.00 |
| | 10 mg/g PVP | 0.33 | 0.01 |
| | 20 mg/g PVP | 0.52 | 0.02 |
| | 50 mg/g PVP | 0.95 | 0.02 |
| | 100 mg/g PVP | 1.33 | 0.01 |
| | 200 mg/g PVP | 1.57 | 0.04 |
| Avicel ™ | No additive | 0.00 | 0.00 |
| | 10 mg/g PVP | 0.00 | 0.00 |
| | 50 mg/g PVP | 0.00 | 0.00 |
| | 100 mg/g PVP | 0.00 | 0.00 |
| Filter paper | No additive | 0.00 | 0.00 |
| | 10 mg/g PVP | 0.00 | 0.00 |
| | 50 mg/g PVP | 0.00 | 0.00 |
| | 100 mg/g PVP | 0.00 | 0.00 |

The nitrogen content of the samples provided an indication of whether any of the nitrogen-containing PVP (or PVP derivatives) remained in them. The results for the Avicel™ and filter paper indicate that the PVP did not bind to cellulose during the pretreatment process, as it was clearly removed by the subsequent washing step. In the wheat straw samples, however, the PVP survived the washing step, its final concentration increasing in line with its concentration during pretreatment. This suggests that the additive was binding to non-cellulosic components of the biomass (ie most probably to the lignin), and thus confirms the results in Example 9.

### Example 11

This example compares the effects of different pretreatment additives, some of the type used in an embodiment of the present invention.

Wheat straw samples were pretreated using the same basic methodology as in Example 1. The additives tested were (1) bovine serum albumin (BSA), a non-catalytic protein used in prior art hydrolyses; (2) 40 kDa PVP; and (3) an alternative tertiary polyamide, poly(2-ethyl-2-oxazoline) (P2EO), of molecular weight ˜50 kDa. All three additives were sourced from Sigma-Aldrich. A control was pretreated without any additive.

The pretreated samples were washed and then subjected to enzymatic hydrolysis for 73 hours. Percentages of cellulose conversion were measured as in Example 1. The results are shown in Table 11 below.

TABLE 11

| | % cellulose | SD |
|---|---|---|
| Sample | conversion | (n = 3) |
| 11A - pretreated without additive | 38.5 | 0.45 |
| 11B - pretreated with BSA | 40.6 | 0.52 |
| 11C - pretreated with P2EO | 50.5 | 0.84 |
| 11D - pretreated with PVP | 58.2 | 0.31 |

It can be seen that the BSA (sample 11B) had no appreciable effect on cellulose conversion when added during the pretreatment step. This was probably due to denaturation of the protein at the high pretreatment temperature. Both the polyamides were effective as pretreatment additives and significantly enhanced the enzymatic hydrolysis, the PVP performing better than the P2EO in this regard.

### Example 12

This example compares the effects of further potential pretreatment additives, some of the type used in an embodiment of the present invention.

Example 11 was repeated using as polymeric pretreatment additives (1) PVP (average molecular weight 40 kDa), (2) P2EO (average molecular weight 50 kDa), (3) a branched polyethylenimine (average molecular weight 25 kDa), (4) poly(4-vinylpyridine) (average molecular weight 60 kDa), (5) chitin (molecular weight ˜400 kDa) and (6) a polyacrylamide (average molecular weight 10 kDa). All were sourced from Sigma-Aldrich, apart from the polyacrylamide which was sourced from Polysciences. The test additives were used at a concentration of 100 mg of additive per gram of biomass. Again a control was pretreated without any additive.

The pretreated samples were washed and then subjected to enzymatic hydrolysis for 72 hours, using Accellerase™ 1000 at a concentration of 5 mg of enzyme per gram of cellulose. Percentages of cellulose conversion were measured as in Example 1; all experiments were performed in triplicate. The results are shown in Table 12 below, which also indicates some of the properties of the test additives.

TABLE 12

| | | | | Mean | |
|---|---|---|---|---|---|
| | Test | Additive functional | Water | % cellulose | SD |
| Sample | additive | group(s) | soluble?* | conversion | (n = 3) |
| 12A | No additive | — | — | 12.67 | 0.08 |
| 12B | PVP | Tertiary amide | Yes | 23.53 | 0.29 |
| 12C | P2EO | Tertiary amide | Yes | 20.97 | 0.48 |
| 12D | Branched | Primary, secondary & | Yes | 8.93 | 0.15 |
| | polyethylenimine | tertiary amines | | | |
| 12E | Poly(4- | Tertiary amine | Yes** | 8.69 | 0.07 |
| | vinylpyridine) | | | | |
| 12F | Chitin | Secondary amide | No*** | 12.45 | 0.35 |
| 12G | Polyacrylamide | Primary amide | Yes | 10.55 | 0.20 |

*Polymers were deemed to be water-soluble if they could be solubilised at 1% w/v in water or in 1% sulphuric acid
**Poly(vinylpyridine) exhibits low solubility in water at neutral pH but is soluble in dilute acid solutions.
***Chitin is described as being soluble in dilute acid. However it exhibited little
solubility in this work.

It can be seen from Table 12 that both of the tertiary polyamide additives improved the efficiency of the subsequent enzymatic hydrolysis. The improvement was not, however, observed with other types of polyamine or polyamide additives, including the secondary amide. It appears that a specific type of additive is needed in order to obtain the benefits of the present invention.

The above examples show that the invention can increase the efficiency of a process for converting biomass to fermentable sugars. This can in turn be of value in an overall process for the production of bioethanol, in particular for use in or as, or for conversion to, a biofuel such as biodiesel or biogasoline. The invention may be used to reduce the overall cost of such a process, for example by allowing it to be carried out under less severe conditions, and/or for a shorter period, and/or with a smaller amount of a reagent such as an enzyme catalyst, without undue detriment to its yield. A processing step which can be carried out under less severe conditions can often be conducted in smaller scale and/or less complex (and thus typically less expensive) equipment, and/or by less skilled operators.

The invention can therefore improve the feasibility of producing bioalcohols directly from biomass.

### Example 13

This example compares the use of tertiary polyamides as an additive with additives mentioned in the prior art. For this example, ball and milled wheat straw particles were slurried in dilute sulphuric acid (1% v/v) with a ratio of 1 g of wheat straw to 9 mL of the aqueous acid solution. Where appropriate, the additive was added to this slurry in an amount as listed in tables 13A and 13B. The pretreatment was performed at 120° C. for 1 hour, in an autoclave. After pretreatment, the substrate was filtered, and washed in water to remove soluble material.

Subsequently an enzymatic hydrolysis was carried out on the pretreated wheat straw using the Accellerase™ 1000 enzymes (ex Sigma-Aldrich) at a pH of 5.0 and a temperature of 50° C. and the glucose concentrations were determined by HPLC in a manner essentially as mentioned herein before.

In table 13A and table 13B, PVP is a polyvinyl pyrrolidone; Tween™ 20 is a polyoxyethylene derivate of sorbitan monolaurate; Tergitol™ is a secondary alcohol ethoxylate; and Triton™ is a polyethylene glycol p-(1,1,3,3-tetramethylbutyl)-phenyl ether.

TABLE 13A
Grams glucose per liter formed when using 10 milligrams of the
additive listed per gram of wheat straw in the pretreatment.

| Time (hours) | | | | | |
|---|---|---|---|---|---|
| of enzymatic | No | | | Tween ™ | |
| hydrolysis | additive | PVP | Tergitol ™ | 20 | Triton ™ |
| 0 | 0.2 | 0.3 | 0.3 | 2.3 | 2.2 |
| 1 | 3.2 | 4.7 | 4.5 | 4.0 | 4.9 |
| 2 | 5.4 | 8.7 | 7.9 | 7.3 | 7.6 |
| 4 | 7.9 | 12.5 | 11.3 | 11.1 | 12.2 |
| 6 | 9.8 | 16.4 | 15.5 | 13.8 | 14.9 |
| 24 | 18.8 | 27.2 | 21.3 | 21.1 | 22.5 |
| 48 | 22.8 | 33.5 | 25.7 | 25.0 | 25.7 |
| 72 | 23.7 | 36.4 | 31.2 | 28.5 | 31.7 |
| 144 | 28.3 | 44.8 | 34.6 | 33.4 | 35.8 |

TABLE 13B
Grams glucose per liter formed when using 100 milligrams of the
additive listed per gram of wheat straw in the pretreatment.

| Time (hours) | | | | | |
|---|---|---|---|---|---|
| of enzymatic | No | | | Tween ™ | |
| hydrolysis | additive | PVP | Tergitol ™ | 20 | Triton ™ |
| 0 | 0.2 | 0.2 | 0.3 | 0.3 | 0.3 |
| 1 | 3.7 | 4.4 | 3.5 | 3.4 | 3.7 |
| 2 | 5.5 | 7.3 | 5.6 | 5.6 | 6.0 |
| 4 | 8.4 | 11.0 | 9.1 | 8.6 | 9.2 |
| 6 | 10.4 | 13.7 | 10.5 | 11.0 | 11.4 |
| 24 | 18.4 | 33.2 | 30.2 | 27.8 | 30.2 |
| 48 | 22.7 | 42.3 | 39.3 | 34.5 | 40.2 |
| 72 | 24.9 | 48.4 | 43.9 | 41.2 | 45.0 |
| 144 | 28.1 | 50.1 | 50.2 | 46.9 | 50.9 |

Example 13 illustrates that the PVP is a more active additive than comparative additives. When using a high amount of additive (100 milligrams of additive per gram of wheat straw) similar results are obtained for the different additives, but when using a lower amount of additive (10 milligrams of additive per gram of wheat straw) PVP outperforms the other additives. In addition, example 13 shows that at shorter operating times for the enzymatic hydrolysis, the amount of glucose formed is higher for the PVP pretreated wheat straw than for the wheat straw pretreated with the other additives.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as the presently preferred embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

What is claimed is:

1. A method for processing a lignocellulosic biomass material, the method comprising:
pretreating a biomass material in the presence of a tertiary polyamide additive, said biomass material comprising one or more cellulosic components; and
subjecting the pretreated biomass material to enzymatic hydrolysis of the one or more cellulosic components to produce a sugar.

2. The method of claim 1, wherein the pretreating step is carried out at a temperature of from about 100 to 200° C.

3. The method of claim 1, wherein the pretreating step comprises contacting the biomass material with an acid.

4. The method of claim 1, wherein the tertiary polyamide additive is an amorphous polymer.

5. The method of claim 1, wherein the tertiary polyamide additive exists in an amorphous form during at least a portion of at least one step of the method.

6. The method of claim 1, wherein the tertiary polyamide additive is an amphiphilic polymer.

7. he method of claim 1, wherein the tertiary polyamide additive is a polymer having one or more amphiphilic molecular regions.

8. The method of claim 1, wherein the tertiary polyamide additive is selected from the group consisting of polyvinyl pyrrolidones, poly(alkyl oxazolines), and any combination thereof.

9. The method of claim 1, wherein the molecular weight of the tertiary polyamide is from about 5 to 100 kDa.

10. The method of claim 1 further comprising the step of:
inducing fermentation of the produced sugar.

11. The method of claim 10, wherein the fermentation produces an alcohol.

12. The method of claim 10 further comprising the step of:
incorporating a product of the fermentation into a biofuel or biofuel component.

13. The method of claim 12 further comprising the step of:
modifying the fermentation product to make it suitable for use in or as a biofuel.

14. The method of claim 13 wherein the modifying step comprises combining the fermentation product with one or more additional fuel components to produce a fuel formulation.

15. The method of claim 1 wherein the amount of at least one other additive is reduced as compared to a method carried out in absence of the tertiary polyamide additive.

16. The method of claim 1 wherein at least one operating condition is less severe as compared to a method carried out in absence of the tertiary polyamide additive.

17. The method of claim 15, wherein the at least one other additive is selected from the group consisting of a surfactant, a substances having an ethylene oxide group, a protein, a nitrogen-containing compound, and any combination thereof.

18. The method of claim 15, wherein the at least one other additive is an additive configured to be used in the enzymatic hydrolysis process.

19. The method of claim 1, wherein the pretreating step is carried out at a pressure of from about 0.05 to 5 MPa.

20. The method of claim 1, wherein the tertiary polyamide additive is in a concentration of at least 0.1% w/w.

21. The method of claim 1 wherein the hydrolysis step further comprises adding more tertiary polyamide additive to the biomass material.
